(19) United States
(12) Patent Application Publication
(10) Pub. No.: US 2016/0051587 A1
(43) Pub. Date: Feb. 25, 2016

(54) INTERNAL MEDICINE DOSING OF STEM CELLS

(71) Applicant: JEREMY DELK, Nicholasville, KY (US)

(72) Inventor: JEREMY DELK, Nicholasville, KY (US)

(21) Appl. No.: 14/832,660

(22) Filed: Aug. 21, 2015

Related U.S. Application Data
(60) Provisional application No. 62/040,153, filed on Aug. 21, 2014.
(60) Provisional application No. 62/040,149, filed on Aug. 21, 2014.
(60) Provisional application No. 62/040,170, filed on Aug. 21, 2014.

(51) Int. Cl. A61K 35/28; A61K 35/19; A61K 35/16; A61K 9/00

(52) U.S. Cl. CPC A61K 35/28; A61K 9/0019; A61K 35/19; A61K 35/16; A61K 2035/124

(57) ABSTRACT

The present invention relates to methods of improving stem cell delivery to a subject in need thereof and kits designed to assist in such. The methods comprise site specific delivery of between five and fifteen million stem cells at the site and systemic administration of between about three and ten million cells per kilogram of the subject receiving the treatment.

### RELATED APPLICATIONS

This patent application claims priority to U.S. Provisional Patent Application Nos. 62/040,149, filed on Aug. 21, 2014, 62/040,153, filed on Aug. 21, 2014, and 62/040,170, filed on Aug. 21, 2014, the entireties of which are incorporated herein by reference.

### FIELD OF THE INVENTION

The present invention relates generally to methods of growing and using stem cells for medical applications.

### BACKGROUND

Recently, it has been proposed to use stem cells to treat bone, ligament, tendon or cartilage injury (see U.S. patent application Ser. No. 13/773,869, incorporated herein by reference in its entirety). Stem cells may be derived from a variety of sources including adipose tissues from the Stromal Vascular Fraction (SVF), bone marrow, the umbilical cord, and blood. Additionally, adipose-derived stem cells (ADSCs) or adipose-derived mesenchymal stem cells (ADMSCs) have been shown to possess the ability to generate multiple tissues, including bone, fat, cartilage, and muscle despite being in an “inactive” state when extracted. As set forth in U.S. patent application Ser. No. 13/77,869, these stem cells can be activated by photo-activation and/or contact with platelet rich plasma (PRP). Thus, adipose tissue has been proposed as an optimal source for adult stem cells (ASCs) for use in regenerative medicine. But, what are needed in the art are improved methods for growing and administering the stem cells.

### SUMMARY OF THE INVENTION

The present invention provides a method of treating a patient, comprising administering autologous, allogeneic or xenogeneic stem cells utilizing platelet rich plasma and photo-biostimulation to activate the stem cells.

The present invention also provides a method of using stem cells to treat a patient, comprising preparing a stem cell preparation; calculating a dosage of said stem cells necessary to treat said patient; and dosing said patient with said dosage. The stem cell preparation may include adipose-derived stem cells or umbilical cord-derived stem cells. The dosage administered is to be proportional to the body weight of said patient. For site specific delivery, the total number of cells administered is between about 5 and 15 million cells. System administration requires administration of between 3 and 10 million cells per kilogram of the subject. The stem cell preparation may additionally include platelet rich plasma to assist in activating the cells and/or already activated stem cells. The dosage may be of around 3-10 million activated stem cells per kilogram of said body weight of said patient total, optionally with 5-15 million of those cells being applied specifically at the site in need thereof. Photo-biostimulation may further be administered to activate the stem cells.

The present invention also provides in part a method of using stem cells to treat a bone, ligament, tendon or cartilage injury in an animal or subject in need thereof, comprising preparing a stem cell preparation; determining an injection site to be treated; calculating a volume of stem cells needed for said injection site; forming a suspension from said stem cell preparation; and injecting said suspension into said injection site. The stem cell preparation may include mesenchymal stem cells and/or adipose-derived stem cells. The stem cell preparation may include previously activated stem cells.

The method may further include selecting a treatment dose. The treatment dose is selected based upon the weight and size of said animal and/or based upon the location of said injection site. The method may further comprise administering a particular dose at the injection site, e.g., intra-articular injection, and administering any remaining suspension intravenously, intrperitonealy, topically and/or intramuscularly, such as any remaining cells required for a dose based on the weight and/or size of the subject. The injection site may be a site of injury or in need of repair within a subject. The method may include preserving any remaining cell suspension for future applications. The method of using stem cells to treat a bone, ligament, tendon or cartilage injury in an animal, may comprise preparing a stem cell preparation; determining sites to be treated; calculating a volume of stem cells needed for all sites; forming a suspension from said stem cell preparation; and applying said suspension to said sites.

### BRIEF DESCRIPTION OF THE DRAWINGS

FIG. 1 shows a 4 year-old spayed, female Cockapoo with Atopy before, during and post administration of stem cells.

### DESCRIPTION

### Stem Cell Administration

The present invention provides for administering stem cells to a patient or a subject. A subject may be a human, canine, feline, bovine, ovine, equine or porcine or a zoo animal. In certain situations, the stem cells are autologous to the patient receiving them. However, certain aspects of the present invention provide methods for preparing allogeneic or xenogeneic stem cells that are administered to the patient or subject.

Autologous and allogeneic cells (or donor cells or non-autologous cells) refer to cells that are genetically different but belong to or are obtained from the same species; autologous cells (or patient cells) are cells that are genetically the same or derived from the same subject or the same subject's same tissue. Xenogeneic cells refer to cells derived from a different species. Stem cells can be deemed allogeneic when administered to a genetically different environment from the source of the cells, such as that of a different patient or subject. Stem cells can be collected and concentrated as described in U.S. patent application Ser. No. 13/773,869 and thereafter, concentrated stem cells may be further activated with isolated platelet rich plasma (PRP) autologous to the patient or subject and may be additionally photo-biostimulated as described in U.S. patent application Ser. No. 13/773,869 in order to activate the cells prior to administration. For the purposes described herein, stem cells may refer to stem cells that are pre-treated with PRP and optionally photo-activated, as well as naïve concentrated stem cells. As described in U.S. patent application Ser. No. 13/773,869, PRP can optionally be prepared from the same sample from which the stem cells are concentrated.

In order to prepare stem cells for administration, a collected fat or other tissue sample from the patient or an allogeneic or xenogeneic subject is treated to isolate the stem cells and optionally PRP from the subject/patient (autologous or allogeneic) to receive the stem cells (optionally photo-activated) which can be mixed with the stem cells and then administered to the patient. The stem cells may be administered systemically, such as by i.v., i.p., i.m., or site-specifically, such as by i.a. As described herein, the administered cells may comprise a specific dose or number of stem cells.

The present invention provides in part for site specific administration of concentrated stem cells, such as the autologous, allogeneic or xenogeneic stem cells discussed herein. Site specific refers to administering stem cells directly at the site in need of repair or treatment, such as a particular joint, bone or lesioned area. Application of the stem cells can be through any known means in the art, such as intra-articularly, intravenously, intraperitonealy, topically, intramuscularly, or suspended in a biocompatible matrix material such as hydroxyapatite, alginate, hyaluronic acid, or collagen or peptide hydrogels, for example. Those skilled in the art will appreciate that topical administration of cells may also be site specific, such as to assist in a particular area of diseased/inflamed skin.

Administration to site specific locations may also comprise administering stem cells pre-incubated in a bioreactor and/or mixed with stem cells cultured in a bioreactor or similar to achieve 3D growth.

Stem cells have not been traditionally been considered for treating internal organs or structures, but instead limited to being administered to tissue from which they were derived. The methods of the present invention may therefore be broadly described as dosing a patient or a subject with concentrated stem cells, either from an autologous, allogeneic or xenogeneic source. Such stem cells may be, e.g., adipose-derived stem cells (ADSCs) or mesenchymal stem cells. The method may include administering a particular dose or range of stem cells to the patient, the dosage or range being determined based upon the patient's body weight. In a particular embodiment, the dosage includes administering about 10 million cells per kilogram of body weight or of at least 3 million/kg. The dose may increase to 4, 5, 6, 7, 8, 9, 10, 11, 12, 13, 14, 15, 16, 17, 18, 19, 20, 30, 40, 50, 60, 70, 80, 90 or 100 million cells per kg. Those skilled in the art will appreciate that subjects failing to respond may require additional cells and/or a higher dose of cells. The method may include administering a particular dose of cells to a site in need thereof, such as intra-articular administration of between about 5 and 10 million stem cells total.

In particular, internal administration of stem cells can be applicable to subjects suffering from inflammatory disorders. For example, inflammatory bowel disease (IBD) involves the chronic inflammation of all or part of a digestive tract. IBD primarily includes ulcerative colitis and Crohn's disease. IBD can be painful and debilitating, and sometimes leads to life-threatening complications. Colitis refers to an inflammation of the colon and is often used to describe an inflammation of the large intestine (colon, caecum, and rectum). Colitis may be acute and self-limited or chronic, i.e. persistent, and broadly fits into the category of digestive diseases. Crohn's disease, also known as Crohn's syndrome and regional enteritis, is a type of IBD that may affect any part of the gastrointestinal tract from mouth to anus. Symptoms often include abdominal pain, diarrhea (which may be bloody if inflammation is severe), fever and weight loss. Other complications may occur outside the gastrointestinal tract and include: anaemia, skin rashes, arthritis, inflammation of the eye, and fatigue. The skin rashes may be due to infections as well as pyoderma gangrenosum or erythema nodosum. Bowel obstruction also commonly occurs and those with the disease are at a greater risk of bowel cancer.

With regard to topical administration, the present invention provides for specific doses or cell number administered to the skin. Those skilled in the art will appreciate that topical administration can be in the form of an applied solution or a lotion and further appreciate that applied cells can be better held at a specific site if applied as part of a bandage. As described herein, a bandage comprising nanofibers may provide an additional scaffold for the cells as applied to the skin. For example, atopy/dermatitis, also known as atopic eczema or eczema, is a type of dermatitis, an inflammatory, relapsing, non-contagious and itchy skin disorder. It has been given names like “prurigo Besnier,” “neurodermatitis,” “endogenous eczema,” “flexural eczema,” “infantile eczema,” and “prurigo diathesique.” Atopy is a fairly prevalent allergic skin disease in canines and felines. Around 16% of all dogs are affected by atopy and 10% of cats. Present treatment for atopic dermatitis includes palliative strategies tailored to the individual patient and include a combination of allergen avoidance, dietary management, topical treatments, non-steroidal antipruritic, corticosteroids, antibiotics, antifungals, hypo sensitization and immune modulators.

Topical administration of stem cells offers a means for site-specific delivery of cells in order to modulate the skin inflammation. The amount/dosage/number of administered cells are size and weight specific based on the subject and/or of the site in need of treatment. The number of stem cells administered can be around 10 million/kg of the subject (for systemic administration, e.g. i.v.) and/or between 5 to 15 million cells at the site (e.g. i.a.). The stem cells may be administered in part or wholly at the site in need of treatment. Topical application may further utilize an applied bandage. Use of biocompatible matrix, such as hydrogels or nanofibers within a bandage may further provide a scaffold and orientation for the stem cells. Nanofibers used within a bandage offer a scaffold to administer stem cells. Stem cells may also be pre-cultured in, or combined with pre-cultured cells from, a bioreactor in order to orient them further for 3D growth/differentiation. A portion of an administered dose may be administered distally from the site in need thereof in conjunction with site-specific administration.

The present invention further provides methods for improving treatment of musculoskeletal injuries and/or degenerative bone and joint diseases. Musculoskeletal injuries and degenerative bone and joint diseases can be disabling and debilitating. Those affected can suffer from reduced mobility and range of motion in addition to experiencing discomfort and pain. The joint of an animal refers generally to the location within the body where two bones are attached in close proximity. Joints are arranged to allow movement and provide structural support. In general, the two bones at the joint are separated by layers of articular cartilage on the opposing surfaces of the two joining bones with a synovial cavity containing synovial fluid between the layers of articular cartilage to provide lubrication for movement at the joint. The synovial cavity is further enclosed by a synovial lining surrounding the joint. Additional connective tissues including ligaments hold the bones at the joint together by attachment to the opposing bones. Similarly, tendons further connect muscle to one of the bones to allow for mechanical movement. Many injuries, conditions and diseases involve the joint and surrounding tissue.

Following injury or tissue damage, tissues may attempt to regenerate new functional cells either by division of existing functional cells or by differentiation of stem cells present in the tissue to form new functional cells. However, unwanted scar tissue may also form as part of the normal healing process, which might impair regeneration and elasticity. Stem cell transplants provide a treatment option for degeneration, damage or injury of the joint and other musculoskeletal tissues as their introduced presence can promote tissue regeneration and restore function and performance while also reducing pain and discomfort to the animal. As described herein, injecting particular doses or counts of stem cells at a site requiring treatment offers an approach that improves specificity of treatment.

The present invention provides a method for using stem cells to treat a bone, ligament, tendon or cartilage injury in a human or an animal, such as a mammal; the method provides for increased cellular localization in an area of ailment and an in vivo resorption period of approximately six weeks. The method may include administering between about 3 to 10 million stem cells per kilogram to the subject, either directly at the site, such as by intra-articular (i.a.) administration or in part with intravenous (i.v.), intraperitoneal (i.p.), topical, and/or intramuscular (i.m.) administration. Additionally, the method may further comprise including the steps of: (a) preparing a stem cell preparation, (b) determining an injection site to be treated, (c) calculating a volume of stems cells needed for the injection site, (d) forming a suspension from the stem cell preparation, and (e) injecting the suspension into the injection site. As described in U.S. patent application Ser. No. 13/773,869, the method may include pre-activation of the stem cells by photo-activation and/or through contact with PRP. The injection may be delivered to the injection site by any known method in the art, such as intravenously or intramuscularly. In one embodiment, the stem cell preparation may include adult, mesenchymal stem cells; in a further embodiment, the stem cells are adipose-derived stem cells (ADSCs) or mesenchymal stromal cells. In yet another embodiment, the stem cell preparation includes activated stem cells. The preparing and activating of the stem cell preparation may be by one or more of the methods fully disclosed and supported by U.S. patent application Ser. No. 13/773,869.

In other embodiments, the method includes selecting a treatment dose based upon the weight and size of the animal and the location of the injection site; any remaining suspension is administered intravenously, intramuscularly, topically and/or preserved for future applications.

### Kits

The present invention also provides for kits for executing the methods described herein. The kits may include: devices for collecting a sample of tissue/blood from which stem cells may be obtained and concentrated, a device for promoting 3D growth of a stem cell, such as a bioreactor, antibiotics, antifungals, a device for administering concentrated stem cells, a device for isolating PRP, a photo bio-stimulator, and a device for concentrating stem cells.

### EXAMPLES

Methods

Acute and chronic colitis was induced in mice by adding 3.5% dextran sulfate sodium to their drinking water. Colitic mice were treated with human mesenchymal stromal cells (MSCs) either via intraperitoneal injections or by intravenous injections and clinical signs and mortality were observed, as was the number of Th1 type Tcells (CD4+) and Tregulatory cells (CD4+CD25+) cells in regional lymph nodes.

Results

Some human MSC isolates produced a significant clinical effect on DSS mice with colitis. Intraperitoneal infusions of human MSCs significantly improved the recovery from clinical signs associated with colitis. Human MSCs significantly improved epithelial recovery seen in histopathology associated with colitis, and reduced mortality. Fewer neutrophils were observed and the crypts were larger in DSS mice which responded to MSC treatment.

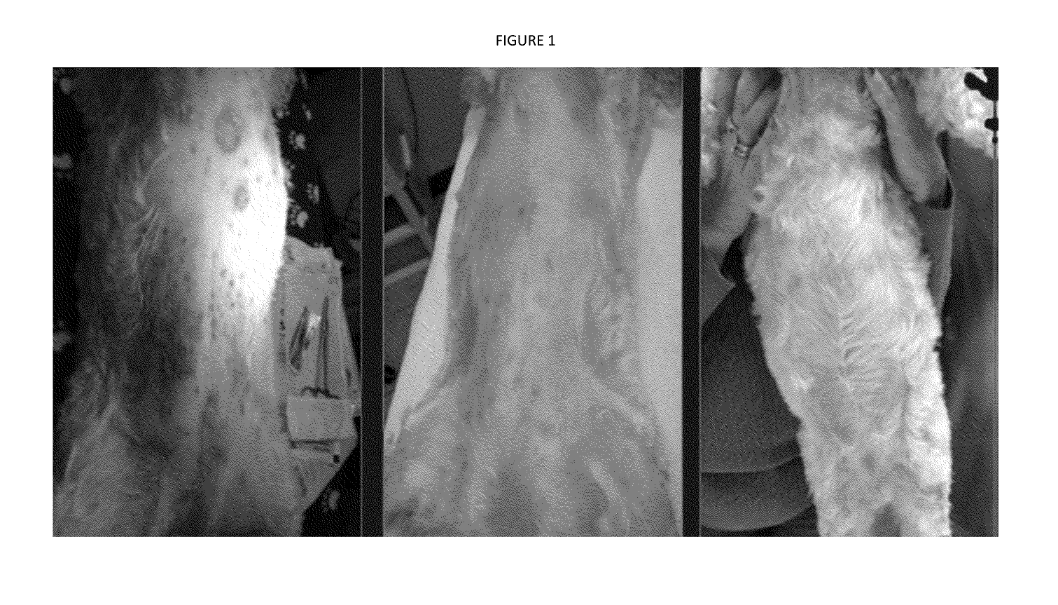

Pictured in FIG. 1 is a 4 year-old spayed, female Cockapoo diagnosed with Atopy; which is a hereditary disorder marked by the tendency to develop immediate allergic reactions to substances such as pollen, food, dander, and insect venoms and manifested by hay fever, asthma, or similar allergic conditions. It is also called atopicallergy. This diagnosis was confirmed after a skin biopsy and skin testing. The dog has a 2-1/2 year history of skin conditions with intense pruritus (itching).

The traditional treatment of steroids, cephalexin and medicated baths were used to manage the dog's pain and suffering. A three month trial with Cyclosporine resulted in GI irritation and had to be discontinued. After the owners elected to try a “compassionate use protocol” of adipose-derived stem cells, their dog was given an intravenous injection of autologous adipose-derived mesenchymal stem cells (FIG. 3). Steroid treatments were stopped 4 weeks prior to adipose collection. The photo on the left shows the dog pre-surgery after being shaved and scrubbed down for fat collection from the falciform region. The picture on the right shows the dog 30 days post-treatment in the same position.

Merely 30 days post-treatment, the dog presents 90% improved from pre-treatment and continues to improve day-to-day. All antibiotics were stopped 10 days after the intravenous treatment of stem cells. Similarly, a 4 year-old neutered, male Irish Setter was diagnosed with Atopy and also treated with a “compassionate use protocol” of autologous adipose-derived mesenchymal stem cells (ADMSCs) administered intravenously. This particular dog was able to live medication-free for 17 months before needing a second treatment of cryogenically banked stem cells. The second treatment is proving to be just as beneficial as the first.

All publications, patents and patent applications references herein are to be each individually considered to be incorporated by reference in their entirety.

What is claimed:

1. A method of treating a site specific injury in a subject comprising administration of a suspension of stem cells to a site in need thereof, wherein the suspension comprises of about between 5 and 15 million cells.

2. The method of claim 1, wherein further cells are administered systemically such that the subject receives between about 3 and 10 million stem cells per kilogram of the subject.

3. The method of claim 1, wherein the stem cells comprise autologous, allogeneic or xenogeneic cells incubated in a bioreactor prior to administration to the subject.

4. The method of claim 1, wherein the suspension further comprises a bio-compatible matrix.

5. The method of claim 1, further comprising mixing the stem cells with autologous platelet rich plasma prior to administration to the subject.

6. The method of claim 4, wherein the stem cells are further photo bio-stimulated.

7. A method of treating a patient, comprising administering a suspension of stem cells to the patient, wherein the suspension of stem cells are derived from autologous stem cells derived from the patient and contacted with autologous platelet rich plasma and/or photo bio-stimulated and administered at or near the site in need.

8. The method of claim 6, wherein the stem cells are administered at a count of between about 3 to 10 million cells per kilogram of the patient intravenously or about 5 to 15 million cells intra-articularly at a joint, tendon or bone in need thereof.

9. A method of using stem cells to treat a bone, ligament, tendon or cartilage injury in an animal, comprising:
preparing a stem cell preparation;
determining an injection site to be treated;
calculating a total number of stem cells needed for said injection site, wherein said total number is equivalent to at least about 10 million activated stem cells per kilogram of the animal;
forming a suspension from said stem cell preparation;
optionally contacting the suspension with photo-biostimulated PRP; and
injecting said suspension into said injection site.

10. The method of claim 8, wherein said stem cell preparation includes mesenchymal stem cells.

11. The method of claim 8, wherein said stem cell preparation includes adipose-derived stem cells.

12. The method of claim 8, wherein said suspension includes a bio-compatible matrix.

13. The method of claim 8, further comprising administering any remaining isolated stem cells from the patient intravenously.

14. The method of claim 8, further comprising administering any remaining isolated stem cells from the patient intramuscularly.

15. The method of claim 8, further comprising preserving any remaining cells isolated from the patient for future applications.
